(19) United States
(12) Patent Application Publication
(10) Pub. No.: US 2017/0086710 A1
(43) Pub. Date: Mar. 30, 2017

(54) ACTIVITY AMOUNT MEASURING APPARATUS, ACTIVITY AMOUNT MEASURING METHOD, AND ACTIVITY AMOUNT MEASURING PROGRAM

(71) Applicant: OMRON HEALTHCARE CO., LTD., Muko-shi (JP)

(72) Inventors: Takehiro HAMAGUCHI, Kyoto (JP); Mika EZOE, Kyoto (JP); Yumi KITAMURA, Kyoto (JP)

(73) Assignee: OMRON HEALTHCARE CO., LTD., Muko-shi (JP)

(21) Appl. No.: 15/372,708

(22) Filed: Dec. 8, 2016

Related U.S. Application Data
(63) Continuation of application No. PCT/JP2015/068499, filed on Jun. 26, 2015.

(30) Foreign Application Priority Data
Jul. 7, 2014 (JP) 2014-139850

(51) Int. Cl. A61B 5/11; A61B 5/00; A63B 24/00

(52) U.S. Cl. CPC A61B 5/1118; A63B 24/0062; A61B 5/6801; A61B 5/0004; A61B 5/742; A63B 2220/40; A63B 2220/74; A63B 2220/73; A63B 2225/50; A63B 2220/836; A63B 2220/17; A61B 2562/0219

(57) ABSTRACT

An apparatus is attached to one portion of a user's body for use, and has a detection unit that detects information corresponding to motion of the user. The apparatus measures an activity amount of the user based on information detected by the detection unit and, if multiple apparatuses are attached to the user's body, transmits information to an activity identification unit that identifies an activity being performed by the user based on information detected by the detection units of the attached activity amount measuring apparatuses and information indicating the attachment portions of the attached activity amount measuring apparatuses. The transmitted information is information that is detected by the detection unit and associated with the information indicating the identified attachment portion. The activity amount is corrected based on the activity that is identified.

### TECHNICAL FIELD

The present invention relates to an activity amount measuring apparatus, an activity amount measuring method, and an activity amount measuring program.

### BACKGROUND ART

Recent years have seen the active development of activity amount measuring apparatuses that measure a person's activity amount (number of steps, walking distance, consumed calories, etc.) using motion detection sensors that detect body motion, such as acceleration sensors and angular velocity sensors. Such activity amount measuring apparatuses are generally attached to one portion of the user's body for use.

However, since people perform various activities, there are cases where the activity amount cannot be measured with merely information from motion detection sensors attached to only one place on the user's body.

Patent Literature 1, 2, and 3 disclose methods in which acceleration sensors are attached at multiple locations on the measurement subject's body, and the measurement subject's activity is identified in a detailed manner by using information from the acceleration sensors.

Patent Literature 4 discloses a system in which activity information measuring apparatuses are attached at multiple locations on the measurement subject's body, information measured by the activity information measuring apparatuses is collected by a management apparatus, and the measurement subject's activity is identified in a detailed manner based on the collected information.

### CITATION LIST

### Patent Literature

Patent Literature 1: JP 2002-291723A

Patent Literature 2: JP 2007-54299A

Patent Literature 3: JP 2011-125696A

Patent Literature 4: JP 2004-184351A

### SUMMARY OF INVENTION

### Technical Problem

As disclosed in Patent Literature 1 to 4, a person's activity can be identified in a detailed manner by using multiple motion detection sensors or multiple activity information measuring apparatuses that are attached at different locations on the user's body. Accordingly, it is thought that using the results of this activity identification makes it possible to measure an accurate activity amount for various activities.

However, the systems disclosed in Patent Literature 1 to 4 are all systems that are based on the premise that multiple motion detection sensors or multiple activity information measuring apparatuses are attached at different locations on the user's body for use. Accordingly, the user needs to always attach the motion detection sensors or the activity information measuring apparatuses to their body, and this attachment task is troublesome. Also, since multiple sensors or apparatuses need to be purchased, the system usage cost increases.

On the other hand, when using an activity amount measuring apparatus that is attached at only one location on the user's body, there is a possibility of not being able to measure an accurate activity amount.

For example, if a user who plays golf or tennis wears a pedometer on only their waist, the number of steps will increase depending on the motion of the user's torso while playing a sport, and it is possible that an accurate number of steps will not be calculated.

Also, if a user performs a task in which they only move their hands while sitting down, and a pedometer is attached to only their wrist, it is possible for the number of steps to increase during the task, and it is possible to not be able to obtain an accurate number of steps. If a user riding in an automobile attaches a pedometer to only their waist, it is possible for the number of steps to increase while traveling, and it is possible to not be able to obtain an accurate number of steps.

In this way, depending on the type of activity, it is possible that an accurate activity amount cannot be measured with a single activity amount measuring apparatus. However, for a user who will use the activity amount measuring apparatus while simply going for a walk, it is sufficient to attach a single activity amount measuring apparatus.

It would be very user-friendly if the activity amount measurement precision could be selected according to the user's needs, but conventional activity amount measuring apparatus have been produced based on the assumption of being used on their own, and cannot accommodate this type of use.

The present invention was achieved in light of the circumstances described above, and an object thereof is to provide a very user-friendly activity amount measuring apparatus that can measure an activity amount with a level of precision that meets the user's needs, as well as an activity amount measuring method performed by such an apparatus, and a program.

### Solution to Problem

An activity amount measuring apparatus according to one aspect of the present invention is an activity amount measuring apparatus that is attached to a user's body for use, the activity amount measuring apparatus including: a detection unit that detects information corresponding to motion of the user; a number of apparatuses determination unit that determines whether or not a plurality of activity amount measuring apparatuses are attached to the user's body; an activity amount measuring unit that measures an activity amount of the user based on the information detected by the detection unit; an attachment portion identification unit that identifies a portion of the user's body to which the activity amount measuring apparatus is attached; and an information transmission control unit that, if the number of apparatuses determination unit detected that a plurality of activity amount measuring apparatuses are attached to the user's body, transmits activity identification information to an activity identification unit that identifies an activity performed by the user based on information detected by the detection units of the attached activity amount measuring apparatuses and information indicating the attachment portions identified by the attachment portion identification units of the attached activity amount measuring apparatuses, the activity identification information being detected by the detection unit and associated with the information indicating the attachment portion identified by the attachment portion identification unit, wherein the activity amount measured by the activity amount measuring unit is corrected based on the activity identified by the activity identification unit.

An activity amount measuring method according to another aspect of the present invention is an activity amount measuring method performed by an activity amount measuring apparatus that is attached to a user's body for use and has a detection unit that detects information corresponding to motion of the user, the method including: a step of determining whether or not a plurality of activity amount measuring apparatuses are attached to the user's body; a step of measuring an activity amount of the user based on the information detected by the detection unit; a step of identifying a portion of the user's body to which the activity amount measuring apparatus is attached; a step of transmitting, if it is detected that a plurality of activity amount measuring apparatuses are attached to the user's body, activity identification information to an activity identification unit that identifies an activity performed by the user based on information detected by the detection units of the attached activity amount measuring apparatuses and information indicating the attachment portions of the attached activity amount measuring apparatuses, the activity identification information being detected by the detection unit and associated with the information indicating the identified attachment portion; and a step of correcting the activity amount measured in the step for measuring the activity amount, based on the activity identified by the activity identification unit.

A program according to another aspect of the present invention is a program for causing a computer to execute the steps of the above-described measuring method.

### Advantageous Effects of Invention

According to the present invention, it is possible to provide a very user-friendly activity amount measuring apparatus that can measure an activity amount with a level of precision that meets the user's needs, as well as an activity amount measuring method performed by such an apparatus, and a program.

### BRIEF DESCRIPTION OF DRAWINGS

FIG. 1 is a block diagram showing a schematic configuration of an activity amount measuring apparatus 10 for describing an embodiment of the present invention.

FIG. 2 is a diagram showing function blocks of a control unit 2 of the activity amount measuring apparatus 10 shown in FIG. 1.

FIG. 3 is a flowchart for describing operations of the activity amount measuring apparatus 10 shown in FIG. 1.

FIG. 4 is a flowchart for describing a specific example of activity identification processing in step S7 in FIG. 3.

FIG. 5 is a diagram showing an example of the attachment of three activity amount measuring apparatuses 10.

FIG. 6 is a function block diagram of a control unit 2A that is a modified example of the control unit 2 in the activity amount measuring apparatus 10 shown in FIG. 1.

FIG. 7 is a diagram showing a configuration of a smartphone that functions as a master apparatus.

FIG. 8 is a flowchart for describing operations of a system made up of a smartphone 30 and the activity amount measuring apparatus 10 that includes the control unit 2A.

### DESCRIPTION OF EMBODIMENTS

Hereinafter, an embodiment of the present invention will be described with reference to the drawings.

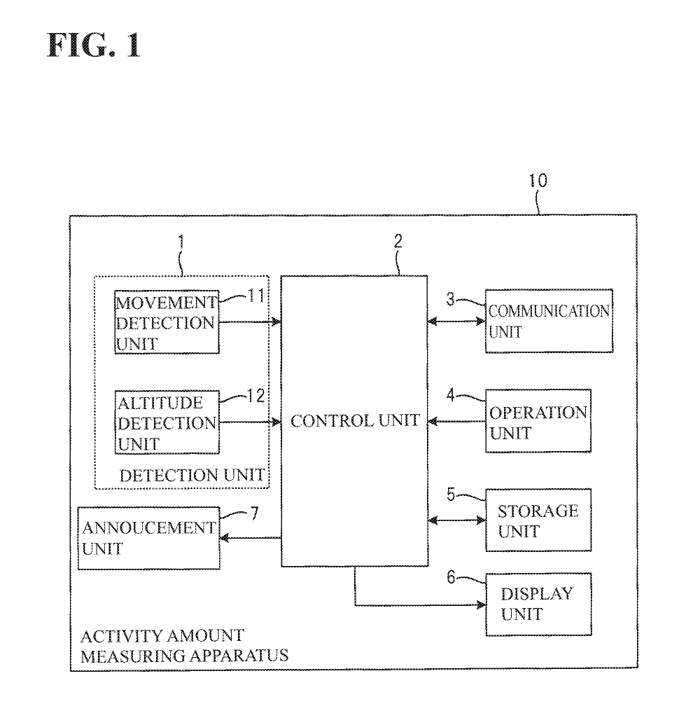

FIG. 1 is a block diagram showing the schematic configuration of an activity amount measuring apparatus 10 for describing an embodiment of the present invention. The activity amount measuring apparatus 10 is attached to a user's body for use. It is possible to use one activity amount measuring apparatus 10 that is attached to the user's body, or use multiple activity amount measuring apparatuses 10 that are attached to the user's body.

The activity amount measuring apparatus 10 includes a detection unit 1, a control unit 2 that performs overall control, a communication unit 3, an operation unit 4, a storage unit 5 that includes a storage medium such as a flash memory or a ROM (Read Only Memory), a display unit 6 for displaying various types of information, and an announcement unit 7 for making an announcement to the user by sound, light, or the like.

The detection unit 1 detects information corresponding to motion of the portion of the user's body to which the activity amount measuring apparatus 10 is attached, and includes a movement detection unit 11 and an altitude detection unit 12.

The movement detection unit 11 detects motion (acceleration, angular velocity, etc.) of the portion of the user's body to which the activity amount measuring apparatus 10 is attached, and includes various sensors such as an acceleration sensor and an angular velocity sensor, and a signal processing unit that processes signals output by the various sensors. It is sufficient that the movement detection unit 11 includes at least one sensor and a signal processing unit that processes signals from that sensor.

The altitude detection unit 12 includes an atmospheric pressure sensor, and detects the altitude of the portion to which the activity amount measuring apparatus 10 is attached by processing a detection signal from the atmospheric pressure sensor. Note that the altitude detection unit 12 may be omitted. The altitude of the portion to which the activity amount measuring apparatus 10 is attached changes depending on the user's activity (e.g., ascending or descending stairs). Accordingly, this altitude information is also information corresponding to motion of the portion to which the activity amount measuring apparatus 10 is attached.

The control unit 2 is mainly constituted by a processor that executes programs stored in the ROM of the storage unit 5.

The communication unit 3 includes an interface for performing communication with other activity amount measuring apparatuses 10. Examples of this interface include a near field communication interface such as an ANT-compliant communication interface or a Bluetooth-compliant communication interface.

The operation unit 4 is a device for inputting various instructions to the control unit 2, and is constituted by buttons, a touch panel installed on the display unit 6, or the like.

The storage unit 5 stores information detected by the detection unit 1, stores activity amounts measured by the control unit 2, stores information received via the communication unit 3, and stores information necessary for operation of the activity amount measuring apparatus 10.

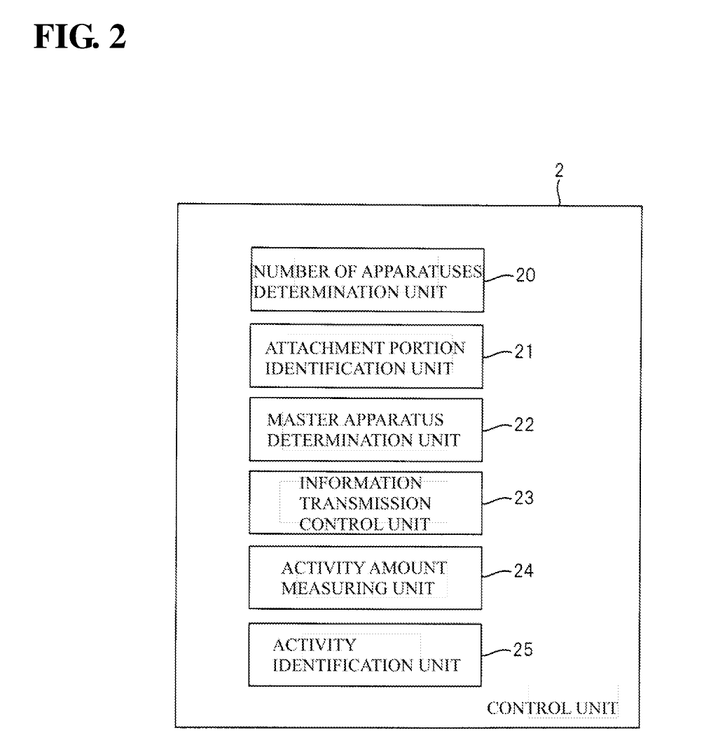

FIG. 2 is a diagram showing function blocks that are realized by the control unit 2 through the processor executing a program stored in the storage unit 5 of the activity amount measuring apparatus 10 shown in FIG. 1.

As shown in FIG. 2, the control unit 2 includes a number of apparatuses determination unit 20, an attachment portion identification unit 21, a master apparatus determination unit 22, an information transmission control unit 23, an activity amount measuring unit 24, and an activity identification unit 25.

The number of apparatuses determination unit 20 determines whether or not multiple activity amount measuring apparatuses 10 are attached to the user's body. Two examples of determination methods are described below, but the present invention is not limited to these two examples.

Number of Apparatuses Determination Method 1

The operation unit 4 can be used to input a state in which only one activity amount measuring apparatus 10 is attached or a state in which multiple activity amount measuring apparatuses 10 are attached. Based on this information input via the operation unit 4, the number of apparatuses determination unit 20 determines whether or not multiple activity amount measuring apparatuses 10 are attached to the user's body.

Number of Apparatuses Determination Method 2

The number of apparatuses determination unit 20 determines that multiple activity amount measuring apparatuses 10 are attached if communication with another activity amount measuring apparatus 10 has been established by the communication unit 3. The number of apparatuses determination unit 20 determines that only one activity amount measuring apparatus 10 is attached if communication with another activity amount measuring apparatus 10 has not been established by the communication unit 3.

The attachment portion identification unit 21 identifies the portion of the user's body to which that activity amount measuring apparatus 10 is attached. Two examples of identifications methods are described below, but the present invention is not limited to these two examples.

Attachment Portion Identification Method 1

The operation unit 4 can be used to select the portion to which the apparatus is attached. The attachment portion identification unit 21 identifies the portion to which the apparatus is attached based on information that indicates the attachment portion and was input via the operation unit 4.

Attachment Portion Identification Method 2

Feature data, which is information indicating the motion of various attachment portions, is stored in the storage unit 5 in advance. After a short while has passed from when multiple activity amount measuring apparatuses 10 were attached, the attachment portion identification unit 21 acquires detection information from the detection unit 1 and specifies the attachment portion by matching acquired detection information pieces with feature data pieces.

If the number of apparatuses determination unit 20 has determined that multiple activity amount measuring apparatuses 10 are attached to the user, the master apparatus determination unit 22 determines one of the activity amount measuring apparatuses 10 that is to be the master apparatus. Three examples of determination methods are described below, but the present invention is not limited to these three examples.

Master Apparatus Determination Method 1

The operation unit 4 can be used to input either “master apparatus” or “slave apparatus” as apparatus attribute information. The master apparatus determination unit 22 then acquires, via the communication unit 3, the attribute information of another activity amount measuring apparatus 10 that was input using the operation unit 4 of that activity amount measuring apparatus 10, and furthermore acquires the attribute information that was input from its own operation unit 4. The master apparatus determination unit 22 then determines which activity amount measuring apparatus 10 is the master apparatus according to all of these pieces of attribute information.

Master Apparatus Determination Method 2

The master apparatus determination unit 22 acquires information indicating the remaining battery amounts of other activity amount measuring apparatuses 10 via the communication unit 3, acquires information indicating its own remaining battery amount, and determines the activity amount measuring apparatus 10 with the highest remaining battery amount as the master apparatus. In other words, the master apparatus determination unit 22 determines the master apparatus based on the remaining battery amounts of all the activity amount measuring apparatuses 10 that are attached to the user.

According to this method, the activity amount measuring apparatus 10 with the highest remaining battery amount becomes the master apparatus, thus making it possible to cause the apparatus that has sufficient battery power to perform tasks that require power, and making it possible to use power resources efficiently. Also, the master apparatus is determined automatically, thus making it possible to eliminate the need for a user operation and to improve the user-friendliness.

Master Apparatus Determination Method 3

Information specifying that the activity amount measuring apparatus 10 attached to a specified portion of the user's body is to be the master apparatus is stored in the storage unit 5 in advance. The master apparatus determination unit 22 compares information indicating the attachment portion identified by the attachment portion identification unit 21 with the information in the storage unit 5, and determines that that activity amount measuring apparatus 10 is the master apparatus if the attachment portion matches the specified portion. On the other hand, if the attachment portion does not match the specified portion, the master apparatus determination unit 22 determines that that activity amount measuring apparatus 10 is a slave apparatus.

The master apparatus determination unit 22 then acquires, via the communication unit 3, the attribute information (master apparatus or slave apparatus) determined by the master apparatus determination units 22 of all of the activity amount measuring apparatuses 10 that are attached to the user, and determines the master apparatus in accordance with the acquired information. In other words, the master apparatus determination unit 22 determines the master apparatus based on the attachment portions of all the activity amount measuring apparatuses 10 that are attached to the user.

According to this method, by setting the wrist as the specified portion for example, the activity amount measuring apparatus 10 with the display unit 6 that is the easiest to view can be set as the master apparatus, and the activity amount measurement results obtained by the master apparatus can be checked easily. Also, the master apparatus is determined automatically, thus making it possible to eliminate the need for a user operation and to improve the user-friendliness.

If the number of apparatuses determination unit 20 determined that multiple activity amount measuring apparatuses 10 are attached to the user's body, the information transmission control unit 23 transmits, to the activity identification unit 25 of the activity amount measuring apparatus 10 that was determined to be the master apparatus, activity identification information in which detection information detected by the detection unit 1 is associated with information indicating the attachment portion identified by the attachment portion identification unit 21.

The information transmission control unit 23 transmits the activity identification information to the included activity identification unit 25 if the activity amount measuring apparatus 10 is the master apparatus. If the activity amount measuring apparatus 10 is a slave apparatus, the information transmission control unit 23 transmits the activity identification information to the activity identification unit 25 of the activity amount measuring apparatus 10 that is the master apparatus via the communication unit 3.

The activity amount measuring unit 24 measures a user activity amount (e.g., number of steps) based on detection information from the detection unit 1, and stores the activity amount. The activity amount measurement result obtained by the activity amount measuring unit 24 can be presented to the user by being displayed on the display unit 6 or being transferred to an external device. If the type of activity is identified by the later-described activity identification unit 25, the activity amount measuring unit 24 performs processing for correcting the measured activity amount based on the activity identification result.

In this specification, the term “activity” refers to states of human actions such as walking, running, sitting, standing still, ascending or descending stairs, and sleeping. For example, ascending or descending stairs can be identified based on change in altitude information detected by the altitude detection unit 12. States such as walking, running, sitting, standing still, and sleeping can be identified based on information detected by the movement detection unit 11.

If the number of apparatuses determination unit 20 determines that multiple activity amount measuring apparatuses 10 are attached to the user's body, the activity identification unit 25 identifies the user activity based on detection information from the detection units 1 of the attached activity amount measuring apparatuses 10, and the associated information indicating the portions to which the apparatuses are attached. Note that the activity identification unit 25 operates only if the activity amount measuring apparatus 10 is the master apparatus. The activity identification result obtained by the activity identification unit 25 can be presented to the user by being displayed on the display unit 6.

The following describes operations of the activity amount measuring apparatus 10 having the configuration described above.

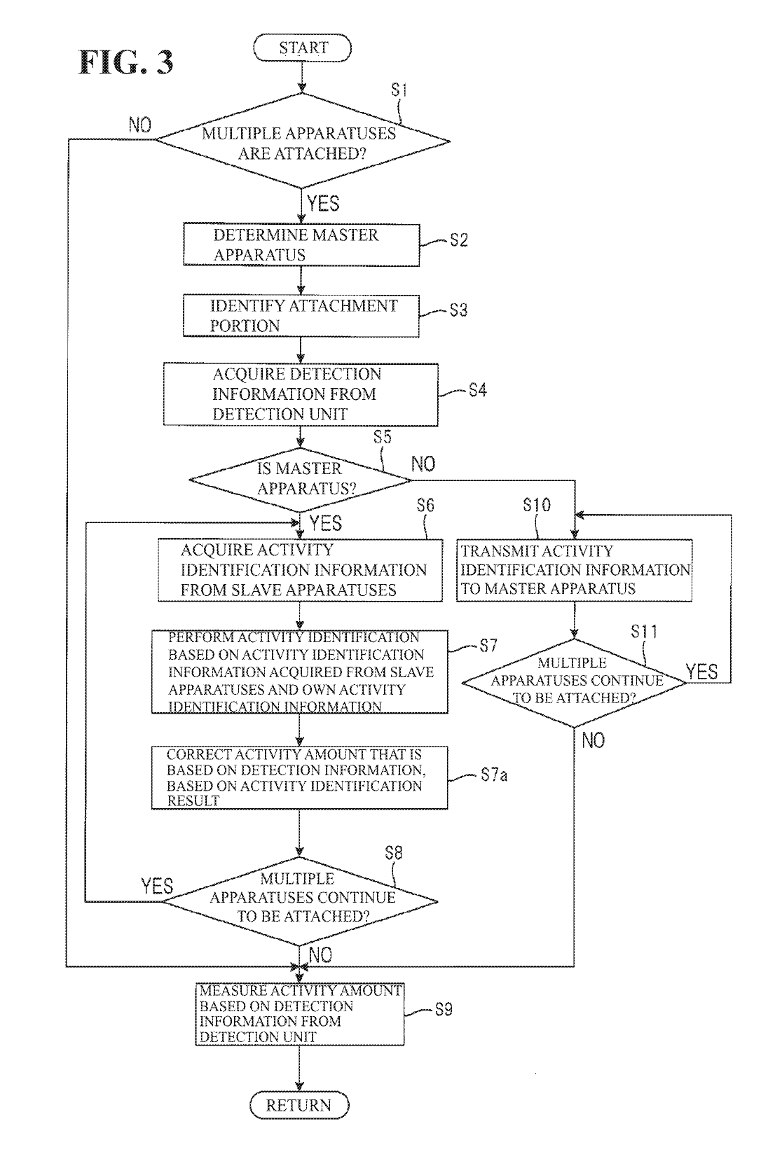

FIG. 3 is a flowchart for describing operations of the activity amount measuring apparatus 10 shown in FIG. 1.

When the power supply of the activity amount measuring apparatus 10 is turned on, the control unit 2 determines whether or not multiple activity amount measuring apparatuses 10 are attached to the user to which that activity amount measuring apparatus 10 is attached (step S1). If the result of the determination in step S1 is NO, the control unit 2 measures an activity amount based on detection information from the detection unit 1 of that activity amount measuring apparatus 10 and records the measured activity amount (step S9).

However, if the result of the determination in step S1 is YES, the control unit 2 determines which one of the activity amount measuring apparatuses 10 that are attached to the user is to be the master apparatus (step S2). In the activity amount measuring apparatus 10 that is determined to be the master apparatus, the control unit 2 may announce to the user that that activity amount measuring apparatus 10 is the master apparatus by operating the announcement unit 7.

Next, the control unit 2 identifies the attachment portion to which the activity amount measuring apparatus 10 is attached (step S3), and acquires detection information from the detection unit 1 (step S4).

Next, if the result of the determination in step S2 is that the activity amount measuring apparatus 10 is the master apparatus (step S5: YES), the control unit 2 performs the processing of step S6.

In step S6, the control unit 2 acquires, from the other activity amount measuring apparatuses 10 that are attached to the user (slave apparatuses), activity identification information that includes detection information obtained by the detection units 1 of the activity amount measuring apparatuses 10 and attachment portion information obtained by the attachment portion identification units 21 of the activity amount measuring apparatuses 10, via the communication unit 3.

The control unit 2 then identifies the user activity based on the activity identification information acquired from the slave apparatuses and the activity identification information generated by that activity amount measuring apparatus 10 (step S7). A specific example of an activity identification method will be described later. Subsequently, based on the activity identification result obtained in step S7, the control unit 2 corrects the activity amount that is based on the detection information from the detection unit 1 (step S7a). For example, if the number of steps is measured as the activity amount, depending on the identified activity, the number of steps measured during the period of that activity may be set to zero by the control unit 2. For example, if the activity is upper body exercise, torso exercise, or lower body exercise, the number of steps measured during that activity is set to zero, whereas if the activity is full body exercise, the number of steps measured during that activity is recorded as-is.

After step S7a, the control unit 2 determines whether or not multiple activity amount measuring apparatuses 10 continue to be attached to the user's body similarly to step S1, and, if multiple activity amount measuring apparatuses 10 continue to be attached (step S8: YES), returns to the processing of step S6. If multiple activity amount measuring apparatuses 10 do not continue to be attached, that is to say if the situation has changed to the case where only one activity amount measuring apparatus 10 is attached to the user's body (step S8: NO), the control unit 2 performs the processing of step 59. In other words, the activity amount is not corrected based on the activity identification result.

If the result of the determination in step S5 is NO, the control unit 2 transmits activity identification information that includes the detection information obtained by the detection unit 1 and the attachment portion information obtained by the attachment portion identification unit 21 to the activity amount measuring apparatus 10 that was determined to be the master apparatus, via the communication unit 3 (step S10).

After step S10, the control unit 2 determines whether or not multiple activity amount measuring apparatuses 10 continue to be attached to the user's body similarly to step S1, and, if multiple activity amount measuring apparatuses 10 continue to be attached (step S11: YES), returns to the processing of step S10. If multiple activity amount measuring apparatuses 10 do not continue to be attached, that is to say if the situation has changed to the case where only one activity amount measuring apparatus 10 is attached to the user's body (step S11: NO), the control unit 2 performs the processing of step S9,

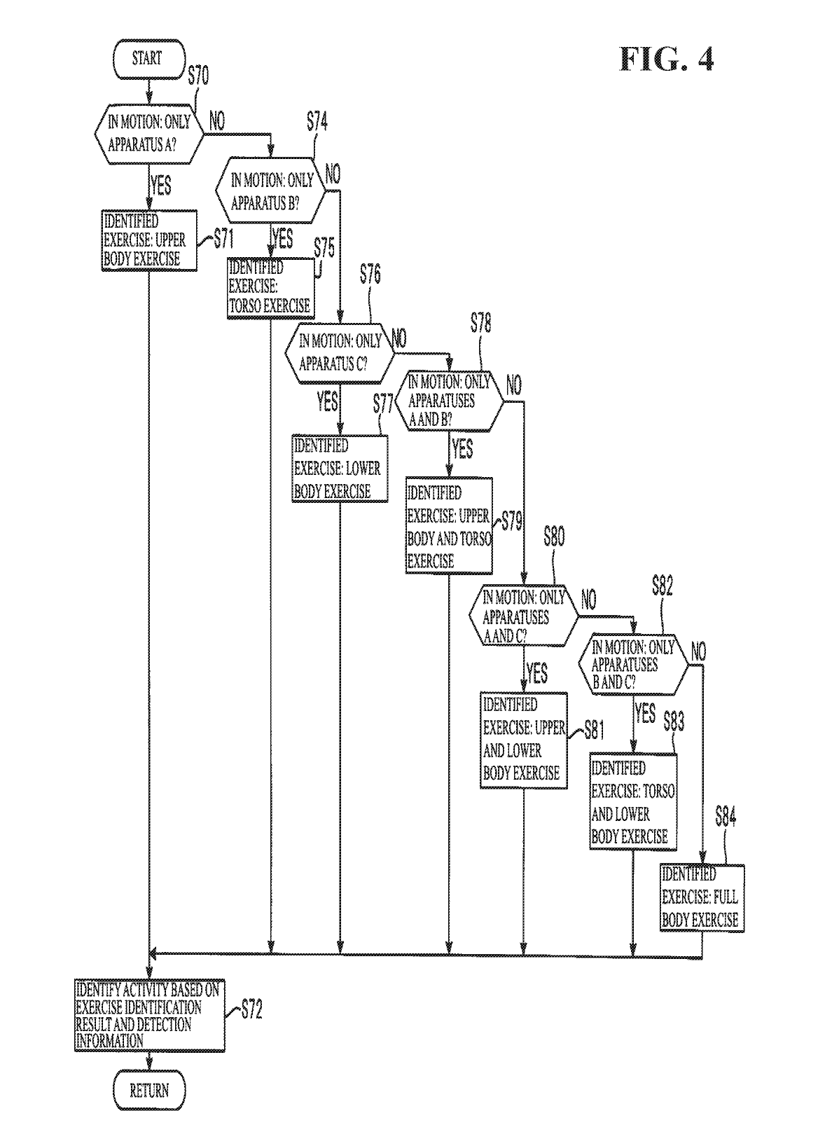

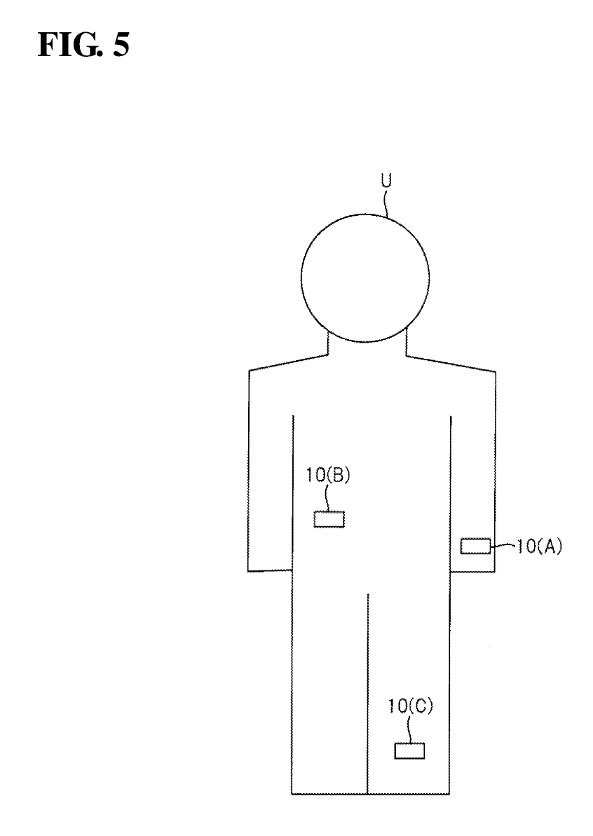

FIG. 4 is a flowchart for describing a specific example of activity identification processing in step S7 in FIG. 3. FIG. 4 shows processing in the state in which activity amount measuring apparatuses 10 are attached to the wrist, waist, and ankle of a user U, as shown in FIG. 5. Hereinafter, the activity amount measuring apparatus 10 attached to the wrist in FIG. 5 will be referred to as “apparatus A”, the activity amount measuring apparatus 10 attached to the waist will be referred to as “apparatus B”, and the activity amount measuring apparatus 10 attached to the ankle will be referred to as “apparatus C”.

The control unit 2 of the master apparatus monitors the detection information obtained by the apparatuses A, B, and C, and determines the apparatus that has the largest amount of change in the detection information to be the apparatus that is in motion. Note that which apparatus each piece of detection information was obtained from can be known based on the attachment portion information that is associated with the piece of detection information.

The control unit 2 determines whether or not the apparatus in motion is only the apparatus A (step S70), and if the result of the determination in step S70 is YES, determines that the user is doing an upper body exercise (step S71).

If the result of the determination in step S70 is NO, the control unit 2 determines whether or not the apparatus in motion is only the apparatus B (step S74). If the result of the determination in step S74 is YES, the control unit 2 determines that the user is doing a torso exercise (step S75).

If the result of the determination in step S74 is NO, the control unit 2 determines whether or not the apparatus in motion is only the apparatus C (step S76). If the result of the determination in step S76 is YES, the control unit 2 determines that the user is doing a lower body exercise (step S77).

If the result of the determination in step S76 is NO, the control unit 2 determines whether or not the apparatuses in motion are only the apparatuses A and B (step S78). If the result of the determination in step S78 is YES, the control unit 2 determines that the user is doing an upper body and torso exercise (step S79).

If the result of the determination in step S78 is NO, the control unit 2 determines whether or not the apparatuses in motion are only the apparatuses A and C (step S80). If the result of the determination in step S80 is YES, the control unit 2 determines that the user is doing an upper body and lower body exercise (step S81).

If the result of the determination in step S80 is NO, the control unit 2 determines whether or not the apparatuses in motion are only the apparatuses B and C (step S82). If the result of the determination in step S82 is YES, the control unit 2 determines that the user is doing a torso and lower body exercise (step S83).

If the result of the determination in step S82 is NO, the apparatuses in motion are the apparatuses A, B, and C, and therefore the control unit 2 determines that the user is doing a full body exercise (step S84).

After performing the exercise identification in step S71, step S75, step S77, step S79, step S81, step S83, and step S84, the activity is identified in detail (step S72) based on the exercise identification results and the detection information generated by the activity amount measuring apparatuses 10 that were determined to be in motion.

For example, if the exercise identification result is “upper body exercise”, it is possible to identify an activity such as “computer work” or “dish washing” based on the motion of the hand indicated by the detection information obtained by the detection unit 1 of the apparatus A. In this case, assuming that the apparatus A is the master apparatus, the number of steps measured in the period of that activity is corrected to zero by the activity amount measuring unit 24 of the apparatus A.

Also, if the exercise identification result is “lower body exercise”, it is possible to identify an activity such as “knee shaking” based on the motion of the ankle indicated by the detection information obtained by the detection unit 1 of the apparatus C. In this case, assuming that the apparatus A is the master apparatus, the number of steps measured in the period of that activity is corrected to zero by the activity amount measuring unit 24 of the apparatus A.

Also, if the exercise identification result is “upper body and torso exercise”, it is possible to identify an activity such as “hanging laundry” based on the motion of the waist and hand indicated by the detection information obtained by the detection units 1 of the apparatuses A and B. In this case, assuming that the apparatus A is the master apparatus, the number of steps measured in the period of that activity is corrected to zero by the activity amount measuring unit 24 of the apparatus A.

Also, if the exercise identification result is “torso and lower body exercise”, it is possible to identify an activity such as “weaving” based on the motion of the waist and foot indicated by the detection information obtained by the detection units 1 of the apparatuses B and C. In this case, assuming that the apparatus A is the master apparatus, the number of steps measured in the period of that activity is corrected to zero by the activity amount measuring unit 24 of the apparatus A.

Also, if the exercise identification result is “upper body and lower body exercise”, it is possible to identify an activity such as “playing the electronic organ” based on the motion of the foot and hand indicated by the detection information obtained by the detection units 1 of the apparatuses A and C. In this case, assuming that the apparatus A is the master apparatus, the number of steps measured in the period of that activity is corrected to zero by the activity amount measuring unit 24 of the apparatus A.

Also, if the exercise identification result is “full body exercise”, it is possible to identify an activity such as “walking” or “running” based on the motion of the waist, hand, and foot indicated by the detection information obtained by the detection units 1 of the apparatuses A, B, and C. In this case, assuming that the apparatus A is the master apparatus, the number of steps measured in the period of that activity is recorded as-is by the activity amount measuring unit 24 of the apparatus A.

As described above, according to the activity amount measuring apparatus 10, when only one activity amount measuring apparatus 10 is attached to the user, a user activity amount is measured and recorded based on detection information obtained by the detection unit 1 of that one activity amount measuring apparatus 10. However, when multiple activity amount measuring apparatuses 10 are attached to the user, one of the activity amount measuring apparatuses 10 becomes the master apparatus, and the master apparatus identifies the user activity based on activity identification information generated by each of the multiple activity amount measuring apparatuses 10. The activity amounts that are based on the detection information from the detection units 1 are then corrected according to the activity identification result. For this reason, activity amount measurement can be performed with a higher precision than in the case where only one activity amount measuring apparatus 10 is attached.

In this way, by allowing the user to select whether to attach one or multiple activity amount measuring apparatuses 10 to their body, it is possible to perform activity amount measurement with a level of precision that meets the user's needs. Multiple activity amount measuring apparatuses 10 only need to be purchased by users who perform activities other than walking and running, and all users do not need to purchase multiple activity amount measuring apparatuses 10, thus making it possible to lower the financial burden on users.

Also, even users who own multiple activity amount measuring apparatuses 10 can select whether to use one or more apparatuses depending on the activity on that day, and it is not necessary to always attach multiple activity amount measuring apparatuses 10. Accordingly, it is possible to prevent the attachment of the activity amount measuring apparatuses 10 from becoming bothersome.

In the above description, the activity amount measuring apparatuses 10 attached to the user's body all have the same configuration, but it is only necessary that at least the configurations of the function blocks corresponding to the detection unit 1 and the control unit 2 match, and other portions may be different between apparatuses. For example, an activity amount measuring apparatus 10 that is not equipped with the display unit 6 and an activity amount measuring apparatus 10 that has the configuration shown in FIG. 1 may be attached to the user's body.

In this case, a system configuration is preferable in which the activity amount measuring apparatus 10 that is equipped with the display unit 6 is set as the master apparatus. Doing this makes it possible for the master apparatus to be used to immediately check the activity identification result that is based on information from multiple activity amount measuring apparatuses 10, and the activity amount measurement result that has been corrected based on the activity identification result.

Note that even if an activity amount measuring apparatus 10 that is not equipped with the display unit 6 becomes the master apparatus, it is not a problem if the system is configured such that data indicating the activity amount measurement result and activity identification result obtained by the activity amount measuring apparatus 10 is transferred to an electronic device such as a smartphone, and the data can be browsed using a smartphone application. In this way, as long as the activity amount and activity identification results can be browsed on another electronic device, the activity amount measuring apparatuses 10 do not need to include the display unit 6.

As described above, the information transmission control unit 23 of the control unit 2 in the activity amount measuring apparatus 10 is configured to transmit all of the detection information obtained by the detection unit 1 to the activity identification unit 25 of the master apparatus. In a modified example, the information transmission control unit 23 may be configured to extract only information that corresponds to the attachment portion of the activity amount measuring apparatus 10 from the detection information obtained by the detection unit 1 of the activity amount measuring apparatus 10, and transmit the extracted information to the activity identification unit 25 of the master apparatus.

For example, consider the case where activity amount measuring apparatuses 10 are attached to the user's wrist and waist. In this case, it is possible for the hand to make complex movements, and therefore acceleration information and angular velocity information are extracted from the information detected by the detection unit 1 and transmitted to the activity identification unit 25. Also, the waist does not make as complex movements, and therefore only acceleration information is extracted from the information detected by the detection unit 1 and transmitted to the activity identification unit 25.

In this way, by controlling the information that is sent to the activity identification unit 25 in accordance with the attachment portion, it is possible to omit the transmission of needless information. The master apparatus can perform activity identification using the minimum required amount of information, thus making it possible to simplify the activity identification algorithm. Also, the memory capacity of the master apparatus can be reduced.

In the above description, it is assumed that one of the activity amount measuring apparatuses 10 attached to the user becomes the master apparatus and performs activity identification. However, a configuration is possible in which an electronic device other than the activity amount measuring apparatuses 10 (e.g., a smartphone) is set as the master apparatus, and all the activity amount measuring apparatuses 10 function as slave apparatuses. This configuration will be described below.

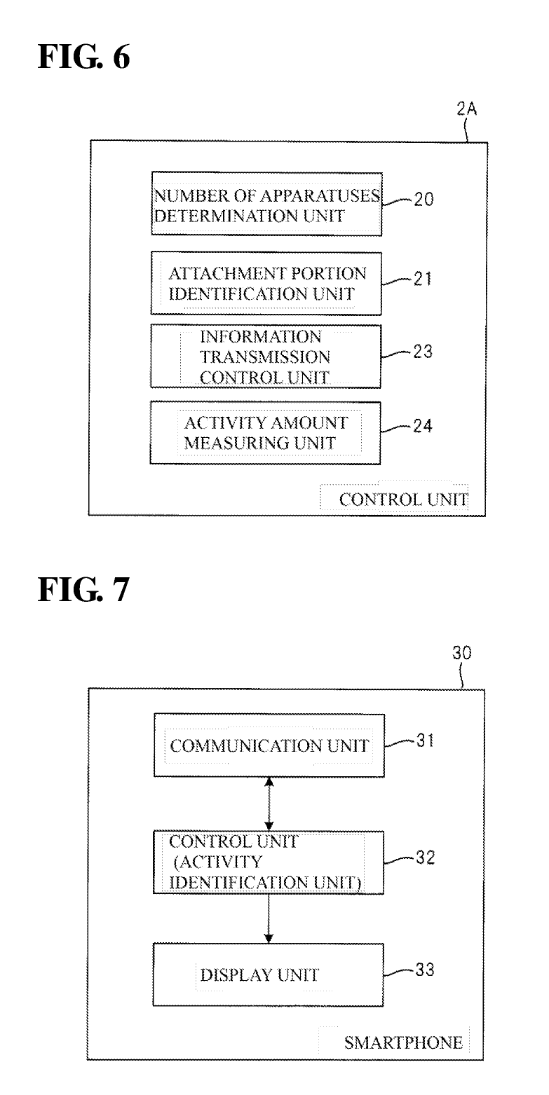

FIG. 6 is a function block diagram of a control unit 2A that is a modified example of the control unit 2 in the activity amount measuring apparatus 10 shown in FIG. 1. The control unit 2A shown in FIG. 6 has the same configuration as the control unit 2 in FIG. 2, with the exception that the master apparatus determination unit 22 and the activity identification unit 25 are omitted.

FIG. 7 is a diagram showing the configuration of a smartphone that is separate from the activity amount measuring apparatuses 10 and functions as the master apparatus. A smartphone 30 includes a communication unit 31, a control unit 32 that is mainly constituted by a processor, and a display unit 33.

The communication unit 31 performs communication with activity amount measuring apparatuses 10 using a communication interface capable of communicating with the activity amount measuring apparatuses 10.

The control unit 32 is mainly constituted by a processor, and functions as the activity identification unit 25 of the control unit 2 in FIG. 2 by the processor executing a predetermined application program.

The display unit 33 is for displaying activity identification results and the like obtained by the control unit 32.

The following describes operations of a system made up of the smartphone 30 and activity amount measuring apparatuses 10 that include the control unit 2A having the configuration described above.

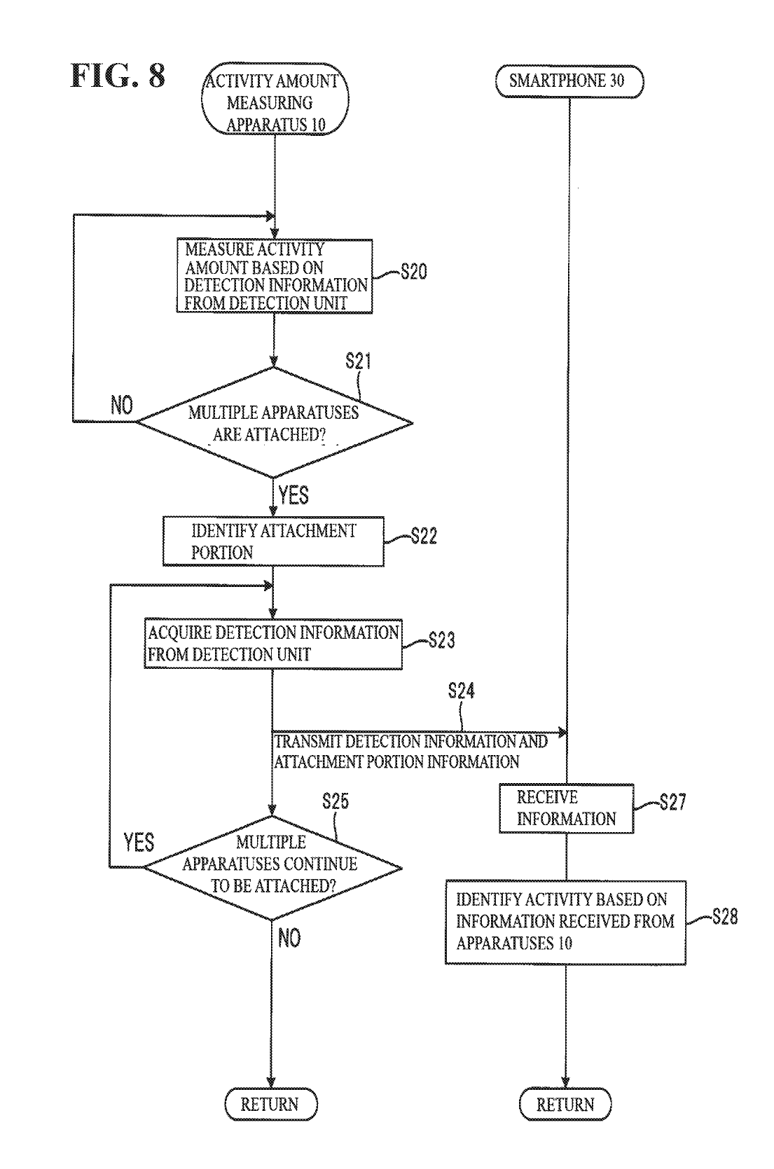

FIG. 8 is a flowchart for describing operations of the system made up of the smartphone 30 and the activity amount measuring apparatuses 10 that include the control unit 2A.

When the power supply of the activity amount measuring apparatus 10 is turned on, the control unit 2A starts the measurement and recording of the activity amount based on detection information obtained by the detection unit 1 (step S20). After the start of measurement, the control unit 2A determines whether or not multiple activity amount measuring apparatuses 10 are attached to the user to which that activity amount measuring apparatus 10 is attached (step S21). If the result of the determination in step S21 is NO, the control unit 2A returns to the processing of step S20.

However, if the result of the determination in step S21 is YES, the control unit 2A identifies the attachment portion of the activity amount measuring apparatus 10 (step S22), and acquires detection information from the detection unit 1 (step S23).

The attachment portion of the activity amount measuring apparatus 10 can be input using an application on the smartphone 30, and in step S22, the activity amount measuring apparatus 10 can identify its attachment portion based on input information that is transmitted from the smartphone 30 to the activity amount measuring apparatus 10.

Next, the information transmission control unit 23 of the control unit 2A transmits, to the smartphone 30, activity identification information that includes the detection information acquired from the detection unit 1 and the information indicating the attachment portion of the activity amount measuring apparatus, via the communication unit 3 (step S24).

The control unit 32 of the smartphone 30 receives the activity identification information via the communication unit 31 (step S27). The control unit 32 then identifies the activity of the user to which the activity amount measuring apparatuses 10 are attached based on the activity identification information received from the activity amount measuring apparatuses 10 (step S28).

After step S24, the control unit 2A determines whether or not multiple activity amount measuring apparatuses 10 continue to be attached to the user's body similarly to step S21, and, if multiple activity amount measuring apparatuses 10 continue to be attached (step S25: YES), returns to the processing of step S23. If multiple activity amount measuring apparatuses 10 do not continue to be attached, that is to say if the situation has changed to the case where only one activity amount measuring apparatus 10 is attached to the user's body (step S25: NO), the control unit 2A returns to the processing of step S20.

When the user ends their exercising or the like, the user gives an activity amount display instruction to the application on the smartphone 30 while one of the activity amount measuring apparatuses 10 and the smartphone 30 are connected. In accordance with this instruction, the control unit 32 of the smartphone 30 acquires measured activity amount information and information indicating the measurement period for the activity amount from the activity amount measuring apparatus 10.

The control unit 32 then corrects the acquired activity amount based on the activity identification result for the acquired period, and displays the corrected activity amount on the display unit 33, For example, if the number of steps measured in one hour by the activity amount measuring apparatus 10 is 1,000, lower body exercise was performed for 30 minutes during that one hour, and full body exercise was performed for the remaining time, the control unit 2A subtracts the number of steps counted in the period corresponding to the lower body exercise from 1,000, and displays the resulting value as the final number of steps.

Although it has been assumed that the smartphone 30 displays a corrected activity amount, a configuration is possible in which the activity identification result is transmitted from the smartphone 30 to the activity amount measuring apparatuses 10, and the activity amount measuring apparatuses 10 display corrected activity amounts based on the activity identification result. Also, a configuration is possible in which the smartphone 30 acquires activity amount information in real-time from the activity amount measuring apparatuses 10 during measurement, corrects the activity amount information based on the activity identification result, and records the corrected activity amount information. Alternatively, a configuration is possible in which the smartphone 30 transmits the activity identification result to the activity amount measuring apparatuses 10 in real-time during measurement, and the activity amount measuring apparatuses 10 correct the activity amounts based on the received activity identification result and record the corrected activity amounts.

As described above, instead of being realized by the activity amount measuring apparatus 10, the functions of the activity identification unit 25 shown in FIG. 2 can be realized by another electronic device that can communicate with the activity amount measuring apparatus 10.

According to this system, it is possible to omit master apparatus determination processing and processing for activity identification based on information from activity amount measuring apparatuses 10 in the activity amount measuring apparatuses 10, thus making it possible to reduce power consumption and extend the battery life of the activity amount measuring apparatuses 10.

The present invention can also be realized as a program for causing a computer to execute the steps performed by the control unit 2, the control unit 2A, and the control unit 32. Such a program can be provided by being recorded on a non-transitory computer-readable recording medium.

Examples of a computer-readable recording medium include optical media such as a CD-ROM (Compact Disc-ROM) and magnetic recording media such as a memory card. Such a program can also be provided by being downloaded via a network.

Note that the embodiment disclosed above is to be understood as being in all ways exemplary and in no way limiting. The scope of the present invention is defined not by the aforementioned descriptions but by the scope of the appended claims, and all changes which come within the meaning and range of equivalency of the scope of the claims are intended to be included therein as well.

As described above, the present specification discloses the following matter.

The disclosed activity amount measuring apparatus is an activity amount measuring apparatus that is attached to a user's body for use, the activity amount measuring apparatus including: a detection unit that detects information corresponding to motion of the user; a number of apparatuses determination unit that determines whether or not a plurality of activity amount measuring apparatuses are attached to the user's body; an activity amount measuring unit that measures an activity amount of the user based on the information detected by the detection unit; an attachment portion identification unit that identifies a portion of the user's body to which the activity amount measuring apparatus is attached; and an information transmission control unit that, if the number of apparatuses determination unit detected that a plurality of activity amount measuring apparatuses are attached to the user's body, transmits activity identification information to an activity identification unit that identifies an activity performed by the user based on information detected by the detection units of the attached activity amount measuring apparatuses and information indicating the attachment portions identified by the attachment portion identification units of the attached activity amount measuring apparatuses, the activity identification information being detected by the detection unit and associated with the information indicating the attachment portion identified by the attachment portion identification unit, wherein the activity amount measured by the activity amount measuring unit is corrected based on the activity identified by the activity identification unit.

In the disclosed activity amount measuring apparatus, among information pieces detected by the detection unit, an information piece that corresponds to the attachment portion identified by the attachment portion identification unit may be transmitted by the information transmission control unit to the activity identification unit as the activity identification information.

The disclosed activity amount measuring apparatus may further include: a communication unit for communicating with another activity amount measuring apparatus; the activity identification unit; and a master apparatus determination unit that, if the number of apparatuses determination unit determined that a plurality of activity amount measuring apparatuses are attached to the user's body, determines an activity amount measuring apparatus that is to be a master apparatus among the plurality of activity amount measuring apparatuses, wherein the information transmission control unit may transmit the activity identification information to the master apparatus via the communication unit if the activity amount measuring apparatus is an activity amount measuring apparatus other than the master apparatus, and if the activity amount measuring apparatus is the master apparatus, the activity identification unit may identify an activity performed by the user based on the activity identification information generated by the activity amount measuring apparatus and the activity identification information received from an activity amount measuring apparatus other than the master apparatus via the communication unit.

In the disclosed activity amount measuring apparatus, the master apparatus determination unit may determine the master apparatus based on a remaining battery amount or the attachment portion of each of the plurality of activity amount measuring apparatuses.

In the disclosed activity amount measuring apparatus, the activity identification unit may be provided in an electronic device that is separate from the activity amount measuring apparatus and can communicate with the activity amount measuring apparatus.

The disclosed activity amount measuring method is an activity amount measuring method performed by an activity amount measuring apparatus that is attached to a user's body for use and has a detection unit that detects information corresponding to motion of the user, the method including: a step of determining whether or not a plurality of activity amount measuring apparatuses are attached to the user's body; a step of measuring an activity amount of the user based on the information detected by the detection unit; a step of identifying a portion of the user's body to which the activity amount measuring apparatus is attached; a step of transmitting, if it is detected that a plurality of activity amount measuring apparatuses are attached to the user's body, activity identification information to an activity identification unit that identifies an activity performed by the user based on information detected by the detection units of the attached activity amount measuring apparatuses and information indicating the attachment portions of the attached activity amount measuring apparatuses, the activity identification information being detected by the detection unit and associated with the information indicating the identified attachment portion; and a step of correcting the activity amount measured in the step for measuring the activity amount, based on the activity identified by the activity identification unit.

The disclosed activity amount measuring apparatus may be realized as a program for causing a computer to execute the steps of the activity amount measuring method described above.

According to the present invention, it is possible to provide a very user-friendly activity amount measuring apparatus that can measure an activity amount with a level of precision that meets the user's needs, as well as an activity amount measuring method performed by such an apparatus, and a program.

While the present invention has been described with reference to a specific embodiment, the present invention is not limited to this embodiment, and many variations and modifications can be made without departing from the technical idea of the present invention.

This application claims the benefit of Japanese Patent Application No. 2014-139850, filed Jul. 7, 2014, which is hereby incorporated by reference herein in its entirety.

### REFERENCE SIGNS LIST

1 Detection unit

2 Control unit

3Communication unit

10 Activity amount measuring apparatus

20 Number of apparatuses determination unit

21 Attachment portion identification unit

22 Master apparatus determination unit

23 Information transmission control unit

24 Activity amount measuring unit

25 Activity identification unit

1. An activity amount measuring apparatus that is attached to a user's body for use, the activity amount measuring apparatus comprising:
a detection unit that detects information corresponding to motion of the user;
a number of apparatuses determination unit that determines whether or not a plurality of activity amount measuring apparatuses are attached to the user's body;
an activity amount measuring unit that measures an activity amount of the user based on the information detected by the detection unit;
an attachment portion identification unit that identifies a portion of the user's body to which the activity amount measuring apparatus is attached; and
an information transmission control unit that, if the number of apparatuses determination unit detected that a plurality of activity amount measuring apparatuses are attached to the user's body, transmits activity identification information to an activity identification unit that identifies an activity performed by the user based on information detected by the detection units of the attached activity amount measuring apparatuses and information indicating the attachment portions identified by the attachment portion identification units of the attached activity amount measuring apparatuses, the activity identification information being detected by the detection unit and associated with the information indicating the attachment portion identified by the attachment portion identification unit,
wherein the activity amount measured by the activity amount measuring unit is corrected based on the activity identified by the activity identification unit.

2. The activity amount measuring apparatus according to claim 1, wherein among information pieces detected by the detection unit, an information piece that corresponds to the attachment portion identified by the attachment portion identification unit is transmitted by the information transmission control unit to the activity identification unit as the activity identification information.

3. The activity amount measuring apparatus according to claim 1, further comprising:
a communication unit for communicating with another activity amount measuring apparatus;
the activity identification unit; and
a master apparatus determination unit that, if the number of apparatuses determination unit determined that a plurality of activity amount measuring apparatuses are attached to the user's body, determines an activity amount measuring apparatus that is to be a master apparatus among the plurality of activity amount measuring apparatuses,
wherein the information transmission control unit transmits the activity identification information to the master apparatus via the communication unit if the activity amount measuring apparatus is an activity amount measuring apparatus other than the master apparatus, and
if the activity amount measuring apparatus is the master apparatus, the activity identification unit identifies an activity performed by the user based on the activity identification information generated by the activity amount measuring apparatus and the activity identification information received from an activity amount measuring apparatus other than the master apparatus via the communication unit.

4. The activity amount measuring apparatus according to claim 3, wherein the master apparatus determination unit determines the master apparatus based on a remaining battery amount or the attachment portion of each of the plurality of activity amount measuring apparatuses.

5. The activity amount measuring apparatus according to claim 1, wherein the activity identification unit is provided in an electronic device that is separate from the activity amount measuring apparatus and can communicate with the activity amount measuring apparatus.

6. An activity amount measuring method performed by an activity amount measuring apparatus that is attached to a user's body for use and has a detection unit that detects information corresponding to motion of the user, the method comprising:
a step of determining whether or not a plurality of activity amount measuring apparatuses are attached to the user's body;
a step of measuring an activity amount of the user based on the information detected by the detection unit;
a step of identifying a portion of the user's body to which the activity amount measuring apparatus is attached;
a step of transmitting, if it is detected that a plurality of activity amount measuring apparatuses are attached to the user's body, activity identification information to an activity identification unit that identifies an activity performed by the user based on information detected by the detection units of the attached activity amount measuring apparatuses and information indicating the attachment portions of the attached activity amount measuring apparatuses, the activity identification information being detected by the detection unit and associated with the information indicating the identified attachment portion; and
a step of correcting the activity amount measured in the step for measuring the activity amount, based on the activity identified by the activity identification unit.

7. A program for causing a computer to execute the steps of the activity amount measuring method of claim 6.

8. The activity amount measuring apparatus according to claim 2, further comprising:
a communication unit for communicating with another activity amount measuring apparatus;
the activity identification unit; and
a master apparatus determination unit that, if the number of apparatuses determination unit determined that a plurality of activity amount measuring apparatuses are attached to the user's body, determines an activity amount measuring apparatus that is to be a master apparatus among the plurality of activity amount measuring apparatuses,
wherein the information transmission control unit transmits the activity identification information to the master apparatus via the communication unit if the activity amount measuring apparatus is an activity amount measuring apparatus other than the master apparatus, and
if the activity amount measuring apparatus is the master apparatus, the activity identification unit identifies an activity performed by the user based on the activity identification information generated by the activity amount measuring apparatus and the activity identification information received from an activity amount measuring apparatus other than the master apparatus via the communication unit.

9. The activity amount measuring apparatus according to claim 8, wherein the master apparatus determination unit determines the master apparatus based on a remaining battery amount or the attachment portion of each of the plurality of activity amount measuring apparatuses.

10. The activity amount measuring apparatus according to claim 2, wherein the activity identification unit is provided in an electronic device that is separate from the activity amount measuring apparatus and can communicate with the activity amount measuring apparatus.
